(19) United States
(12) Patent Application Publication
(10) Pub. No.: US 2015/0267255 A1
(43) Pub. Date: Sep. 24, 2015

(54) METHOD OF DETECTING CHROMOSOMAL ABNORMALITIES

(71) Applicant: Premaitha Health Ltd, London (GB)

(72) Inventors: Charles Edward Selkirk Roberts, London (GB); Robert Old, London (GB); Francesco Crea, London (GB)

(73) Assignee: Premaitha Health Ltd., London (GB)

(21) Appl. No.: 14/424,805

(22) PCT Filed: Aug. 29, 2013
(86) PCT No.: PCT/GB2013/052261

§ 371 Date: Feb. 27, 2015

Related U.S. Application Data
(60) Provisional application No. 61/695,182, filed on Aug. 30, 2012.

(30) Foreign Application Priority Data
Aug. 30, 2012 (GB) 1215449.8

(51) Int. Cl. C12Q 1/68; G06F 19/22

(52) U.S. Cl. CPC C12Q 1/6883; C12Q 1/6879; G06F 19/22; C12Q 2600/156

(57) ABSTRACT

The invention relates to a method of detecting chromosomal abnormalities, in particular, the invention relates to the diagnosis of fetal chromosomal abnormalities such as trisomy 21 (Down's syndrome) which comprises sequence analysis of cell-free DNA molecules in plasma samples obtained from maternal blood during gestation of the fetus.

### FIELD OF THE INVENTION

The invention relates to a method of detecting chromosomal abnormalities, in particular, the invention relates to the diagnosis of fetal chromosomal abnormalities such as trisomy 21 (Down's syndrome) which comprises sequence analysis of cell-free DNA molecules in plasma samples obtained from maternal blood during gestation of the fetus.

### BACKGROUND OF THE INVENTION

Down's Syndrome is a relatively common genetic disorder, affecting about 1 in 800 live births. This syndrome is caused by the presence of an extra whole chromosome 21 (trisomy 21, T21), or less commonly, an extra substantial portion of that chromosome. Trisomies involving other autosomes (i.e. T13 or T18) also occur in live births, but more rarely than T21.

Generally, conditions where there is fetal aneuploidy resulting either from an extra chromosome, or from the deficiency of a chromosome, create an imbalance in the population of fetal DNA molecules in the maternal cell-free plasma DNA that is detectable.

Developing a reliable method for prenatal diagnosis of fetal chromosomal abnormalities has been a long-term goal in reproductive care (Puszyk et al., 2008, Prenat Diagn 28, 1-6). Methods based on obtaining fetal material by amniocentesis or chorionic villus sampling are invasive, and carry a non-negligible risk to the pregnancy even in the hands of skilled clinicians. In current practice, such invasive diagnostic methods are usually employed where there is an indication of an increased chance of a Down's pregnancy, either by reason of maternal age, or through prior screening using biochemical tests or ultrasonography. There is a need for a method of non-invasive prenatal diagnosis (NIPD) that is reliable, applicable in the first trimester, fast in returning a result, and inexpensive.

Progress towards achieving this goal has been made by exploiting the finding that cell-free DNA in the blood plasma of pregnant women includes a component of fetal origin (Lo et al., 1997, Lancet 350, 485-487). The cell-free plasma DNA (referred to hereinafter as ‘plasma DNA’) consists primarily of short DNA molecules (80-200 bp) of which typically 5%-20% are of fetal origin, the remainder being maternal (Birch et al., 2005, Clin Chem 51, 312-320; Fan et al., 2010, Clin Chem 56, 1279-1286). The cellular origins of plasma DNA molecules, and the mechanisms by which they enter the blood and are subsequently cleared from the circulation, are poorly understood. However, it is widely believed that the fetal component is largely the result of apoptotic cell death within the placenta (Bianchi, 2004, Placenta 25, S93-S101). The fraction of the plasma DNA molecules that are of fetal origin varies from case to case with substantial individual variation. Superimposed on the individual variation is a general trend towards an increasing fetal component as gestational age increases (Birch et al., 2005, supra; Galbiati et al., 2005, Hum Genet 117, 243-248). The fetal component is readily detectable early in gestation, typically as early as week 8.

In principle, if the cell-free fetal DNA in plasma were undiluted by the maternal component, the extra chromosome that characterises T21 would be expected to cause a 50% excess of DNA molecules derived from that chromosome, by comparison with a normal pregnancy. However, taking a typical value of 10% for the component of cell-free plasma DNA that is of fetal origin, the imbalance that results is expected to be only 5%, or a relative increase in the number of chromosome 21-derived fragments to a value of 1.05 relative to 1.00 for a normal pregnancy. In situations where the fetal component of the plasma DNA is smaller or larger than the 10% value, the imbalance in the number of chromosome 21-derived molecules in the population of molecules in maternal plasma will be correspondingly smaller or larger.

Therefore, the basis of a diagnostic test for T21 is to obtain nucleotide sequence data (‘DNA sequencing’) for DNA molecules from maternal plasma. Once partial or complete nucleotide sequence information has been obtained from individual DNA molecules, bioinformatic techniques must be applied to assign, most simply by comparison with a reference human genome or genomes, individual molecules to chromosomes from which they originate. In cases of pregnancy involving a fetus with T21, a slight imbalance in the population of molecules is detectable as an excess in the number of chromosome 21-derived molecules over that expected from a normal pregnancy.

In view of the fact that chromosome 21 comprises only a small fraction of the human genome (less than 2%), in order to collect a large enough number from that chromosome for reliable diagnosis, a large number of DNA molecules from maternal plasma must be randomly sampled, sequenced, and assigned bioinformatically to particular chromosomes. The total number of plasma DNA molecules required to be both (1) characterised by nucleotide sequence information derived from them, and then (2) reliably assigned to chromosomal locations, is smaller than that required to sample all or most of the fetal genome, but it is at least several hundred thousand molecules. The minimal number required is a function of the fraction of the plasma DNA that makes up the fetal component of the population of maternal cell-free plasma DNA molecules. Typically the number is between one million or several million molecules.

The challenge of applying this method is considerable because of the high quantitative accuracy required in counting DNA molecules from particular chromosomal locations. Furthermore, the DNA from maternal plasma is a mixture of genomes within which the fetal component is a small part. This quantitative technical problem is different in nature from identifying mutations at a particular locus within a DNA sample.

Given that some nucleotide sequence data can be obtained for sufficiently large numbers of plasma DNA, and given that bioinformatic methods can be reliably applied to assign a sufficiently large number to their chromosomal origin, statistical methods may be applied to determine the presence or absence of a chromosomal imbalance in the population of plasma DNA molecules with statistical confidence.

This idea of sequencing a random sample of DNA fragments from maternal plasma, but the sample making up only a fraction of the complete genome, is the basis of NIPD methodology described in Fan et al., 2008, Proc Natl Acad Sci USA 105, 16266-16271 and Chiu et al., 2008, Proc Natl Acad Sci USA 105, 20458-20463.

Previous diagnostic methodology in this field has exploited massively-parallel DNA sequencing technology that yields high quality sequence data, relatively free of errors, to obtain sequences of sufficient length to be assigned to their chromosomal origin. A significant disadvantage of those methods known to date, which make use of massively-parallel sequencing (also known as next generation sequencing or second generation sequencing) for this purpose, is that the sequencing being performed is at high quality on full-service genome sequencers—predominantly the Illumina HiSeq—which generate very voluminous data requiring time-consuming and expensive bio-informatics. The run time and analysis process may take several weeks in aggregate. A further disadvantage is that the capital outlay of these devices is significant (well in excess of half a million dollars at the present time) which limits widespread access to them. Furthermore, the capacity to multiplex is limited, tying up these expensive machines and further limiting access to rapid throughput diagnostics for large numbers of patients. However, such is the clinical need for non-invasive prenatal diagnosis that even these range of disadvantages have not prevented the beginnings of deployment of massively-parallel sequencing.

However, certain automated sequencing devices typically generate sequence data that is of a quality that is substantially less good than that required for conventional genome sequencing. The sequence data so generated is characterised by frequent errors. These errors are of various kinds, but most commonly are very frequent ‘indels’, that is errors caused by the sequencing device delivering false extra bases (insertions) or deleted bases. In addition there is an inherent inability to sequence short homopolymer runs (i.e. runs of several identical bases) effectively. Furthermore, sequencing errors may also include ‘mismatches’ wherein a base is incorrectly assigned.

This ‘economy grade’ sequencing is of the kind produced inexpensively and rapidly by some benchtop high throughput sequencers, such as the Ion Torrent sequencing platform. This sequencing platform is based upon semiconductor sequencing technology (Rothberg et al., 2011, Nature 475, 348-352). When a nucleotide is incorporated into a growing DNA chain in a polymerase-catalysed reaction, a proton is released. By detecting the associated change in pH, the technology detects whether a nucleotide has been added or not. The semiconductor chip is flooded sequentially with one of the four DNA nucleotide precursors (dATP, dCTP, dGTP or dTTP). If a nucleotide is not incorporated into the growing chain, no voltage is generated; if two nucleotides are added, the voltage change is approximately double. Sequencing homopolymer runs of bases is problematical as the homopolymer length increases. Indel errors (false base insertion or deletion) are frequent, particularly being associated with homopolymer runs.

Before a DNA sample can be sequenced, the workflow involves attaching specific adapter sequences, and emulsion PCR. The preparation time is typically less than 6 hours, and sequencing runs per se are less than 3 hours. The performance of the Ion Torrent sequencing platform has been reviewed recently, along with other high throughput benchtop sequencers (Loman et al. 2012, Nature Biotechnology 30(5), 434-439; Liu et al. 2012, Journal of Biomedicine and Biotechnology 2012, 1-11; Quail et al. 2012, BMC Genomics, 13(341)). The quality of the sequence data generated by the Ion Torrent device is recognised as characterised by frequent indel errors.

Accurate diagnosis within the field of fetal abnormalities is of critical importance. There is therefore a great need for diagnostic methodology which is tolerant of very frequent insertion or deletion (indel) errors and mishandled short homopolymer runs, typically characterised by certain automated sequencing devices.

### SUMMARY OF THE INVENTION

According to a first aspect of the invention, there is provided a method of detecting a fetal chromosomal abnormality in a biological sample obtained from a female subject, the method comprising the steps of:
(a) obtaining sequence data for nucleic acid molecules within the biological sample;
(b) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(c) measuring the ratio of the total number of matched nucleic acids assigned to a target chromosome relative to the total number of matched nucleic acids assigned to each of one or more reference chromosomes;

wherein a statistically significant difference in the measured ratio, relative to the ratio in a normal pregnancy, is indicative of a fetal abnormality in the target chromosome.

According to a second aspect of the invention there is provided a method of predicting the gender of a fetus within a pregnant female subject, the method comprising the steps of:
(a) obtaining a biological sample from the pregnant female subject;
(b) obtaining sequence data for nucleic acid molecules within the biological sample;
(c) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(d) measuring the ratio of the total number of matched nucleic acids assigned to the Y chromosome relative to the total number of matched nucleic acids assigned to each of one or more reference chromosomes;

wherein the presence of matched Y chromosome sequences above a pre-determined ratio is indicative of the presence of a male fetus and the presence of matched Y chromosome sequences below a pre-determined ratio is indicative of the presence of a female fetus.

### BRIEF DESCRIPTION OF THE FIGURES

FIG. 1: Prinseq Sequence Duplication Summary Statistics. Exemplification of the use of Prinseq in producing concise summary statistics and also, importantly, producing the number of duplicate sequences that are prevalent in the sample the raw data of which is shown in the table below:

| | |
|---|---|
| No of Sequences | Max Duplicates |

| | | | |
|---|---|---|---|
| Exact Duplicates | 205,024 | (10.04%) | 1727 |
| Exact Duplicates with | 7 | (0.00%) | 1 |
| Reverse Complements | | | |
| 5′ Duplicates | 44,507 | (2.18%) | 6 |
| 3′ Duplicates | 8,330 | (0.41%) | 7 |
| 5′/3′ Duplicates with | 2,789 | (0.14%) | 2 |
| Reverse Complements | | | |
| Total | 260,657 | (12.77%) | — |

FIG. 2: Analysis of 27 blood plasma samples according to the method of the invention. FIG. 2 shows Z scores for blood plasma samples from normal pregnancies (samples 1-15) and blood plasma samples from Trisomy 21 pregnancies (samples 16-27).

### DETAILED DESCRIPTION OF THE INVENTION

According to a first aspect of the invention, there is provided a method of detecting a fetal chromosomal abnormality in a biological sample obtained from a female subject, the method comprising the steps of:
(a) obtaining sequence data for nucleic acid molecules within the biological sample;
(b) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(c) measuring the ratio of the total number of matched nucleic acids assigned to a target chromosome relative to the total number of matched nucleic acids assigned to each of one or more reference chromosomes;

wherein a statistically significant difference in the measured ratio, relative to the ratio in a normal pregnancy, is indicative of a fetal abnormality in the target chromosome.

The present invention specifies appropriate bioinformatic processing that is specifically tolerant of very frequent substitution and indel errors and mishandled short homopolymer runs. This bioinformatic processing allows reliable assignment of sequences to chromosomes in an appropriately efficient way i.e. combining reliability without rejecting a practically unworkable, large, fraction of the sequence data as unmatchable to any chromosome, or mis-assigning them to an incorrect chromosomal location.

Chromosome Abnormalities

Examples of suitable chromosomal abnormalities which the invention finds utility in detecting include: Down's Syndrome (Trisomy 21), Edward's Syndrome (Trisomy 18), Patau syndrome (Trisomy 13), Trisomy 9, Warkany syndrome (Trisomy 8), Cat Eye Syndrome (4 copies of chromosome 22), Trisomy 22, and Trisomy 16.

Additionally, or alternatively, the detection of an abnormality in a gene, chromosome, or part of a chromosome, copy number may comprise the detection of and/or diagnosis of a condition selected from the group comprising Wolf-Hirschhorn syndrome (4p-), Cri du chat syndrome (5p-), Williams-Beuren syndrome (7-), Jacobsen Syndrome (11-), Miller-Dieker syndrome (17-), Smith-Magenis Syndrome (17-), 22ql l.2 deletion syndrome (also known as Velocardiofacial Syndrome, DiGeorge Syndrome, conotruncal anomaly face syndrome, Congenital Thymic Aplasia, and Strong Syndrome), Angelman syndrome (15-), and Prader-Willi syndrome (15-).

Additionally, or alternatively, the detection of an abnormality in the chromosome copy number may comprise the detection of and/or diagnosis of a condition selected from the group comprising Turner syndrome (Ullrich-Turner syndrome or monosomy X), Klinefelter's syndrome, 47,XXY or XXY syndrome, 48,XXYY syndrome, 49,XXXXY Syndrome, Triple X syndrome, XXXX syndrome (also called tetrasomy X, quadruple X, or 48,XXXX), XXXXX syndrome (also called pentasomy X or 49,XXXXX) and XYY syndrome.

In one embodiment, the target chromosome is chromosome 13, chromosome 18, chromosome 21, the X chromosome or the Y chromosome.

In one embodiment, the fetal chromosomal abnormality is a fetal chromosomal aneuploidy. In a further embodiment, the fetal chromosomal aneuploidy is trisomy 13, trisomy 18 or trisomy 21. In a yet further embodiment, the fetal chromosomal aneuploidy is trisomy 21 (Down's syndrome). In this embodiment, the skilled worker in the field will readily understand that the methodology of the invention can be applied to diagnosing cases where the fetus carries a substantial part of chromosome 21 rather than an entire chromosome.

Sample Extraction

It will be appreciated that samples may be obtained from a pregnant female subject in accordance with routine procedures. In one embodiment, the biological sample is maternal blood, plasma, serum, urine or saliva. In a further embodiment the biological sample is maternal plasma.

The step of obtaining maternal plasma will typically involve a 5-20 ml blood sample (typically a peripheral blood sample) being withdrawn from the pregnant female subject (typically by venipuncture). Obtaining such a sample is therefore characterised as noninvasive of the fetal space, and is minimally invasive for the mother. Blood plasma is prepared by conventional means after removal of cellular material by centrifugation (Maron et al., 2007, Methods Mol Med 132, 51-63).

DNA is extracted from the maternal plasma by conventional methodology which is unbiased with respect to the nucleotide sequences of the plasma DNA (Maron et al., 2007, supra). The population of plasma DNA molecules will typically comprise a fraction that is of fetal origin, and a fraction of maternal origin.

Obtaining Sequence Data

DNA sequence data for a sufficient number of plasma DNA molecules, at least 500,000 and typically several million molecules (Fan and Quake, 2010, PLoS One 5), is generally obtained and prepared for bioinformatic analysis. The sufficient number will be statistically determined for the type of abnormality to be detected. The bioinformatic analysis is specifically designed to be tolerant of indel and mismatch errors while efficiently extracting the required information in the form of reliable matches to unique sequences of particular chromosomes.

It will be appreciated by the skilled person that the invention is not limited to any particular technique for obtaining the sequence data. However, it is appreciated that the methods of the invention find greater utility when the quality of the sequence data is less optimal than that typically observed for conventional genome sequencing. For example, in one embodiment, the sequence data is obtained by a sequencing platform which comprises use of a polymerase chain reaction. In a further embodiment, the sequence data is obtained using a next generation sequencing platform. Such sequencing platforms have been extensively discussed and reviewed in: Loman et al (2012) Nature Biotechnology 30(5), 434-439; Quail et al (2012) BMC Genomics 13, 341; Liu et al (2012) Journal of Biomedicine and Biotechnology 2012, 1-11; and Meldrum et al (2011) Clin Biochem Rev. 32(4): 177-195; the sequencing platforms of which are herein incorporated by reference.

Examples of suitable next generation sequencing platforms include: Roche 454 (i.e. Roche 454 GS FLX), Applied Biosystems' SOLiD system (i.e. SOLiDv4), Illumina's GAIIx, HiSeq 2000 and MiSeq sequencers, Life Technologies' Ion Torrent semiconductor-based sequencing instruments, Pacific Biosciences' PacBio RS and Sanger's 3730xl.

Each of Roche's 454 platforms employ pyrosequencing, whereby chemiluminescent signal indicates base incorporation and the intensity of signal correlates to the number of bases incorporated through homopolymer reads.

In one embodiment, the sequence data is obtained from a sequencing platform which comprises use of semiconductor-based sequencing methodology. The virtues of semiconductor-based sequencing methodology are that the instrument, chips and reagents are very cheap to manufacture, the sequencing process is fast (although off-set by emPCR) and the system is scalable, although this may be somewhat restricted by the bead size used for emPCR.

In one embodiment, the sequence data is obtained by a sequencing platform which comprises use of sequencing-by-synthesis. Illumina's sequencing-by-synthesis (SBS) technology is currently a successful and widely-adopted next-generation sequencing platform worldwide. TruSeq technology supports massively-parallel sequencing using a proprietary reversible terminator-based method that enables detection of single bases as they are incorporated into growing DNA strands. A fluorescently-labeled terminator is imaged as each dNTP is added and then cleaved to allow incorporation of the next base. Since all four reversible terminator-bound dNTPs are present during each sequencing cycle, natural competition minimizes incorporation bias.

In one embodiment, the sequence data is obtained from a sequencing platform which comprises use of nanopore-based sequencing methodology. In a further embodiment, the nanopore-based methodology comprises use of organic-type nanopores which mimic the situation of the cell membrane and protein channels in living cells, such as in the technology used by Oxford Nanopore Technologies (e.g. Branton D, Bayley H, et al (2008). Nature Biotechnology 26 (10), 1146-1153). In a yet further embodiment, the nanopore-based methodology comprises use of a nanopore constructed from a metal, polymer or plastic material.

In one embodiment, the next generation sequencing platform is selected from Life Technologies' Ion Torrent platform or Illumina's MiSeq. The next generation sequencing platforms of this embodiment are both small in size and feature fast turnover rates but provide limited data throughput.

In a further embodiment, the next generation sequencing platform is a personal genome machine (PGM) which is Life Technologies' Ion Torrent Personal Genome Machine (Ion Torrent PGM). The Ion Torrent device uses a strategy similar to sequencing-by-synthesis (SBS) but detects signal by the release of hydrogen ions resulting from the activity of DNA polymerase during nucleotide incorporation. In essence, the Ion Torrent chip is a very sensitive pH meter. Each ion chip contains millions of ion-sensitive field-effect transistor (ISFET) sensors that allow parallel detection of multiple sequencing reactions. The use of ISFET devices is well known to the person skilled in the art and is well within the scope of technology which may be used to obtain the sequence data required by the methods of the invention (Prodromakis et al (2010) IEEE Electron Device Letters 31(9), 1053-1055; Purushothaman et al (2006) Sensors and Actuators B 114, 964-968; Toumazou and Cass (2007) Phil. Trans. R. Soc. B, 362, 1321-1328; WO 2008/107014 (DNA Electronics Ltd); WO 2003/073088 (Toumazou); US 2010/0159461 (DNA Electronics Ltd); the sequencing methodology of each are herein incorporated by reference).

The SBS chemistry used by both 454 and Ion Torrent is also conducive to longer reads. Ion Torrent is currently restricted to fragments much shorter than that of Roche 454 but this will likely improve with future versions. Both the Roche 454 and Ion Torrent platforms have the common issue of homopolymer sequence errors manifesting as false insertions or deletions (indels). It is believed that Roche will adopt a similar detection method to Ion Torrent through a licence from DNA Electronics which is likely to make the 454 and Ion Torrent platforms essentially identical.

In one embodiment, the sequence data is obtained by a sequencing platform which comprises use of release of ions, such as hydrogen ions. This embodiment provides a number of key advantages. For example, the Ion Torrent PGM is described in Quail et al (2012; supra) as the most inexpensive personal genome machines on the market (i.e. approx. $80,000). Furthermore, Loman et al (2012; supra) describes the Ion Torrent PGM as producing the fastest throughput (80-100 Mb/h) and the shortest run time (˜3 h). However, it is well documented that the Ion Torrent PGM is characterised by frequent indel errors. For example, Loman et al (2012; supra) describes that the Ion Torrent PGM produced the shortest reads and the worst homopolymer-associated indel error rate. The issue of high error rate is further confirmed in a comparison between the Illumina MiSeq and Ion Torrent PGM (http://www.illumina.com/documents/analysis of inaccuracies in ion torrent long read application.pdf) which claims that the MiSeq total error rate is substantially lower than the PGM total error rate. Such disadvantageous properties regarding error rates of Ion Torrent PGM are discussed in independent blog sites such as http://omicsomics.blogspot.co.uk/ and http://pathogenomics.bham.ac.uk/blog/author/nick/

It will be appreciated that later generations of the Ion Torrent device may also find utility in the invention, for example in one embodiment, the sequence data is obtained by multiplex capable iterations based upon the Life Technologies' Ion Torrent platform, such as an Ion Proton with a PI or PII Chip, and further derivative devices and components thereof.

Furthermore, the inventors of the present invention have analysed the number of indels present when performing the step of obtaining sequence data according to the invention with the Ion Torrent PGM and the results are summarised in Table 1:

TABLE 1
Frequency of Molecules Showing Indels

| Total Good | No. Reads | % Reads | No. Reads ≧2 | % Reads ≧2 |
|---|---|---|---|---|
| Hits | with Indels | with Indels | Indels | Indels |
| 1629343 | 959167 | 58.9 | 570564 | 35.0 |
| 2345252 | 1361760 | 58.1 | 782332 | 33.4 |
| 2085793 | 1126147 | 54.0 | 615676 | 29.5 |
| 1158299 | 729292 | 63.0 | 460934 | 39.8 |

Table 1 shows data from four maternal plasma DNA samples and summarises the frequency of molecules possessing 1 or more or 2 or more indels from a set of maternal plasma DNA molecules obtained, sequenced and matched to chromosomal locations, according to the invention. The majority of the mapped sequence reads show at least one indel. These data refer to matched sequence reads (“good hits”) obtained in accordance with the methodology of the present invention.

Thus, it would be clearly apparent to the skilled person that the Ion Torrent platform, or indeed other personal genome machines, would be unsuitable for a critical technique for diagnosing chromosome abnormalities—especially when the results may ultimately determine whether a fetus is terminated or not. By contrast, the Illumina Genome Analyser and more recently the HiSeq 2000 have set the standard for high throughput massively-parallel sequencing (Quail et al. 2012, BMC Genomics, 13(341)), although such devices are more costly and time consuming.

However, the methods of the invention combine the advantageous properties of error prone devices such as the Ion Torrent device (i.e. cost, speed and throughput) with a low stringency matching analysis which surprisingly overcomes the disadvantages with respect to high error rates.

Duplicate Collapsing

Prinseq was employed as a metagenomic tool for monitoring the quality and characteristics of the Ion Torrent PGM sequencing data (Schmieder and Edwards, 2011, Bioinformatics 27, 863-864). It provides summary statistics for the raw sequence data, which relates to base composition, length distributions, base quality calls, di-nucleotide frequencies and duplicate sequences.

As proportions of chromosomal matches are involved in the diagnosis, one important statistic is the number of exact duplicates in the data; in addition, the chance of exact duplicate sequences naturally appearing in the maternal plasmas is low; their occurrence is an unexpected artefact. Therefore, the removal of duplicate sequences by performing a step of exact duplicate sequence collapse is considered an important pre-processing step.

Thus, in one embodiment the methods of the invention additionally comprise the step of collapsing duplicate reads from the sequence data obtained prior to the matching analysis step.

It will be apparent to the skilled person how collapsing duplicate sequences may be performed. For example, the FASTQ/A Collapser software within the FASTX-Toolkit provides the ability to collapse identical sequences into a single sequence while maintaining an accurate number of read counts.

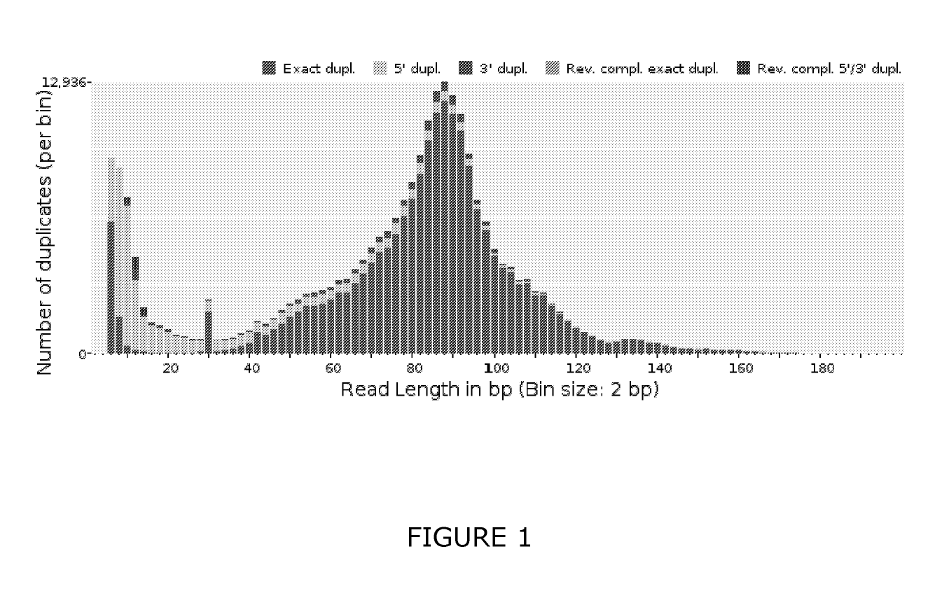

FIG. 1 shows an example of sequence duplication distribution and shows the percentage of the total reads that were duplicates (10% in this particular example). The FASTX-Toolkit was used to collapse exact duplicate sequences (the same sequence over the full length).

Matching Analysis

Previous applications of non-invasive aneuploidy (Chiu et al., 2008, Proc Natl Acad Sci USA 105, 20458-20463) used Solexa/Illumina short read sequencing technology. These reads were all 36 bp in length and they employed a stringent read to genome mapping procedure that attempted to account for genome repeats and copy number variation. They mapped reads to a repeat masked genome and counted reads that only mapped to one position in the genome with 100% identity, over the entire read length.

By contrast, when applying Ion Torrent PGM to the methods of the invention, sequences were generated that were of variable length, from approximately 20 to 260 bp.

Following collapse of exact duplicate reads as described hereinbefore, the method of the invention then conducts a matching analysis. Such a matching analysis typically involves a bioinformatic analysis which is performed on an unmasked reference genome using suitable software.

In one embodiment, the matching analysis is conducted using Bowtie2 or BWA-SW (Li and Durbin (2010) Bioinformatics, Epub) alignment software or alignment software employing Maximal Exact Matching techniques, such as BWA-MEM (lh3lh3.users.sourceforge.net/download/mem-poster.pdf) or CUSHAW2 (http://cushaw2.sourceforge.net/) software. In a further embodiment, the matching analysis is conducted using Bowtie2 software. In a yet further embodiment, the Bowtie2 software is Bowtie2 2.0.0-beta7.

In an alternative embodiment, the matching analysis is conducted using alignment software employing Maximal Exact Matching (MEM) techniques, such as BWA-MEM (lh3lh3.users.sourceforge.net/download/mem-poster.pdf) or CUSHAW2 (http://cushaw2.sourceforge.net/) software. The MEM algorithms are believed to have the advantage of providing greater accuracy

The advantage of using longer read lengths than those used for Solexa/Illumina data, is that reads could be soft clipped and it was found that no repeat masking was required prior to mapping.

For the mapping of sequences to unique chromosomal locations, the indel/mismatch cost weighting must be parameterised to low in this analysis. With these pre-conditions, non-stringent fragment-length matches are determined. Using this bioinformatic approach, typically about 95% of sample reads are mapped to the genome. Reads are only counted as assigned to a chromosomal location if they match to a unique position in the genome, typically bringing the proportion of sample reads uniquely matched and subsequently counted for the chromosomal assignments to about 50%.

It will be appreciated that the matching process most simply uses one or more reference genomes. However, it is envisaged that alternative approaches may be employed, such as a reference-free approach in which accumulated sequence data from many normal and affected pregnancies are analysed to determine which sequences form the set of sequences that are potentially in imbalance for a particular chromosomal abnormality, such as Trisomy 21.

In one embodiment, the matching analysis is conducted with respect to a whole chromosome, for example, the analysis would therefore comprise detecting an excess of a given chromosome. In an alternative embodiment, the matching analysis is conducted with respect to a part of said chromosome, for example, matches will be analysed solely with respect to a particular pre-determined region of a chromosome. It is believed that this embodiment of the invention provides a more sensitive matching technique by virtue of targeting a specific region of a chromosome.

The non-stringent matching analysis of the invention typically involves an alignment scoring system where an accuracy score is assigned for a matching base and penalties are applied for a substitution or mismatch, the presence of an ambiguity (i.e. N) in either the read or reference and the presence of a gap (i.e. insertion or deletion) in the read or reference. Once the score has been calculated for each hit, the score is compared with a minimum alignment score threshold. The scoring system typically used in the invention uses the local alignment scoring example in accordance with the Bowtie2 software.

In one embodiment, the accuracy score assigned for each base within the nucleic acid which corresponds to a base in the reference genome is a positive score. In a further embodiment, a positive score of +2 is assigned for each base within the nucleic acid which corresponds to a base in the reference genome (i.e. the match score is +2). For example, the Bowtie2 software sets a match score of +2 for each position where a read character aligns to a reference character and the characters match. The match score is referred to in the Bowtie2 software as “- − -ma” (or match bonus).

In one embodiment, the penalisation score for any insertions, deletions, ambiguities and/or substitutions is a reduced score, such as a negative score.

In a further embodiment, a negative score of −6 is assigned for a substitution or mismatch (i.e. a mismatch or substitution penalty is −6). For example, a value of 6 is subtracted from the alignment score for each position where a read character aligns to a reference character and the characters do not match (and neither is an N). The mismatch or substitution penalty is referred to in the Bowtie2 software as “- − -mp”.

In one embodiment, the negative score for an ambiguity (N penalty) is −1. For example, a value of 1 is subtracted from the alignment score for positions where the read, reference, or both, contain an ambiguous character such as N. The ambiguity or N penalty is referred to in the Bowtie2 software as “- − -np”.

In one embodiment, the negative score for an insertion or deletion is −5 plus −3 for each residue within the insertion or deletion. In a further embodiment, the gap penalty in the read fragment is −5 for the gap and −3 for each extension within the gap. For example, a “length −2” read gap receives a penalty of −11 in total (i.e. −5 for the gap, −3 for the first extension within the gap and −3 for the second extension within the gap). The gap penalty in the read fragment is referred to in the Bowtie2 software as “- − -rdg”.

In a further embodiment, the gap penalty in the reference fragment is −5 for the gap and −3 for each extension within the gap. The gap penalty in the reference fragment is referred to in the Bowtie2 software as “- − -rfg”.

In one embodiment, the minimum alignment score is calculated in accordance with the following equation:

a+b*ln(L)

wherein a and b refer to scoring parameters determined to optimize matching accuracy and In refers to the natural logarithm of the read length (L).

In a further embodiment, the minimum alignment score is calculated in accordance with the following equation:

20+8.0*ln(L)

wherein In refers to the natural logarithm of the read length (L).

For example, for a read length of 20 bases, the minimum score threshold is 20+8*ln 20=20+8*2.995=20+23.97=43.97. Therefore, a perfect match for a 20 base read length would score 40 which would never reach the minimum score threshold of 43.97 and so a read length of 20 bases will be typically too short to be considered a match.

By contrast, for a read length of 50 bases, the minimum score threshold is 20+8*ln50=20+8*3.91=20+31.3=51.3. Therefore, a perfect match for a 50 base read length would score 100, therefore, a read length of 50 bases would tolerate a few mismatches and indels and still be considered a matching hit.

It will be appreciated that the concept of the minimum alignment score requires shorter read lengths to have less indels and mismatches and permits longer read lengths to have a greater number of indels and mismatches. Thus, in one embodiment, the nucleic acid fragment reads comprise from approximately 25 bp to approximately 250 bp.

It will also be appreciated that other examples of alignment software (i.e. BWA-SW, BWA-MEM and CUSHAW2) operate in an analogous manner to the scoring system described above for Bowtie2.

Therefore, the alignment analysis software described herein (such as Bowtie2, BWA-SW, BWA-MEM and CUSHAW2) is particularly advantageous by virtue of solving the problems of: (1) exact duplicate sequences; (2) homopolymer runs; (3) frequent indel errors; (4) repeat sequences in the genome; and (5) to a large extent, copy number variation.

Ratio Calculation

Once the total number of hits have been assigned to a given chromosome in accordance with the matching analysis herein defined, the hits are then typically normalised to a common number (suitably per 1 million hits). The ratio of each hits for a target chromosome compared with hits on other chromosomes is then calculated in accordance with simple mathematics—an example of which is described herein in Example 1.

In addition to normalization to a common number as referred to hereinbefore, it is typically useful to be able to estimate the fraction of the maternal plasma DNA that is fetal in origin; this will confirm that there is sufficient fetal DNA in a sample of maternal plasma DNA for detecting a fetal chromosomal abnormality. For example, in one embodiment, the method of the invention additionally comprises the step of normalizing or adjusting the number of matched hits based on the amount of fetal DNA within the sample.

Statistical Significance

In order to place the diagnostic test of the invention on a statistical basis, the method of the invention additionally comprises the step of calculating statistical significance of the ratio of each hits for a target chromosome compared with hits on other chromosomes. In one embodiment, the statistical significance test comprises calculation of the z-score in accordance with conventional statistical analysis of the reduced counting data. However, it will be appreciated that other statistical methods may be applied by skilled workers in the field.

Where the distribution of the errors in the counts ratio “target chromosome/other chromosomes” is assumed to be approximately normal, the z-score indicates how many standard deviations an element is from the mean.

A z-score can be calculated from the following formula:

z=(X−μ)/σ

wherein z is the z-score, X is the value of the element, μ is the population mean, and σ is the standard deviation of the population values. When testing for the presence of Trisomy 21 according to the present invention, a z-score value of 2.0 or more for the count ratio indicates a probability of approx 98% that the count ratio value indicates a Trisomy 21 pregnancy.

Methods of Predicting Gender

The presence of Chromosome Y DNA, which is inherited from the paternal parent of the fetus, is a diagnostic marker of a male fetus. A further aspect of the present invention is the detection of the gender of the fetus as indicated by the presence of Chromosome Y sequences.

Where the fetus is female the use of the Y chromosomal component is precluded, however in place of the paternally-inherited Y-chromosome, it is possible to detect gene alleles that are paternally-derived. Among these are fetal SNPs (single nucleotide polymorphisms), which are evident as alleles present as a minor component of the DNA sequences in maternal plasma DNA (Dhallan et al., Lancet 369, 474-481). Where a fraction of the fetal genome only is sequenced, as in the present invention, the number of such alleles inherited from the fetus' father, and detected as variants differing from the relatively more abundant maternal alleles, is a function of the fraction of the plasma DNA that is fetal. This provides an alternative, gender-independent, method for estimating the fraction of maternal plasma DNA that is fetal in origin.

According to a second aspect of the invention there is provided a method of predicting the gender of a fetus within a pregnant female subject, the method comprising the steps of:
(a) obtaining a biological sample from the pregnant female subject;
(b) obtaining sequence data for nucleic acid molecules within the biological sample;
(c) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(d) measuring the ratio of the total number of matched nucleic acids assigned to the Y chromosome relative to the total number of matched nucleic acids assigned to each of one or more reference chromosomes;

wherein the presence of matched Y chromosome sequences above a pre-determined ratio is indicative of the presence of a male fetus and the presence of matched Y chromosome sequences below a pre-determined ratio is indicative of the presence of a female fetus.

In a male pregnancy the quantity of Y-chromosomal material is a measure of the fraction of the plasma DNA that is of fetal origin. Where the fetus is female this measure is not applicable, and other means are adopted to determine the fraction of plasma DNA that is fetal. It will be apparent to the skilled person that alternative paternally-derived allelelic variants that are highly polymorphic, such as short tandem repeats, can be analysed to quantify the fraction of fetal DNA in plasma.

It will be appreciated that all of the embodiments with respect to the detection method of the first aspect of the invention apply equally to the gender prediction method of the second aspect of the invention.

The following study illustrates the invention:

### EXAMPLE 1

### Detection of Trisomy 21 in Blood Plasma Samples

In order to evaluate the effectiveness of the methods of the invention in diagnosing Trisomy 21, blood plasma samples were separately obtained from normal pregnancies and Trisomy 21 pregnancies in accordance with routine procedures (for example a 5-20 ml blood sample was withdrawn from the subject and the plasma was separated followed by extraction of plasma DNA).

The plasma DNA was then subjected to sequence analysis using the Ion Torrent PGM device. For example, adaptors were attached, a library was prepared and emulsion PCR was performed prior to sequence analysis.

The sequence data was then obtained for approx. 25 bp-250 bp for a large number of individual molecules, typically 1-10 million reads.

The data was subjected to bioinformatic analysis as described hereinbefore. For example, duplicate reads were collapsed using the FASTX-Toolkit. The data was then subjected to a matching analysis using Bowtie2 software exactly as described hereinbefore in order to prepare non-stringent fragment length unique matches to the reference genome. Copy number variation was also excluded.

The number of mapped reads and their chromosomal locations are shown in Table 2, for four maternal plasma DNA samples, i.e. two normal (N1 and N2) and two Trisomy 21 pregnancies (T21/1 and T21/2):

TABLE 2

| | N1 | N2 | T21/1 | T21/2 |
|---|---|---|---|---|
| Total Good Hits | 1629343 | 2345252 | 2085793 | 1158299 |
| Ch21 | 20104 | 28878 | 27972 | 14886 |
| Ch1 | 131059 | 188779 | 168275 | 93285 |
| Ch2 | 147717 | 210896 | 188316 | 104095 |
| Ch3 | 116167 | 166072 | 147717 | 82985 |
| Ch4 | 106690 | 147890 | 133298 | 75467 |
| Ch5 | 105237 | 148239 | 131549 | 73341 |
| Ch6 | 99476 | 141651 | 125973 | 71348 |
| Ch7 | 85633 | 122515 | 109511 | 61071 |
| Ch8 | 84523 | 120805 | 106333 | 59806 |
| Ch9 | 65094 | 94167 | 82995 | 46194 |
| Ch10 | 81391 | 119154 | 105788 | 58122 |
| Ch11 | 75751 | 111144 | 98202 | 54242 |
| Ch12 | 74419 | 106053 | 94751 | 52830 |
| Ch13 | 57421 | 80205 | 72327 | 40552 |
| Ch14 | 51107 | 73540 | 65984 | 36252 |
| Ch15 | 53538 | 77903 | 68912 | 37647 |
| Ch16 | 43897 | 65389 | 58024 | 31113 |
| Ch17 | 42431 | 63681 | 56745 | 31022 |
| Ch18 | 47154 | 67288 | 59877 | 33113 |
| Ch19 | 21318 | 32657 | 29586 | 15680 |
| Ch20 | 36439 | 54121 | 47541 | 25777 |
| Ch22 | 18545 | 29055 | 25392 | 13657 |
| ChX | 63286 | 94000 | 79355 | 45203 |
| ChY | 934 | 1165 | 1363 | 608 |
| ChM | 12 | 5 | 7 | 3 |

The data in Table 2 were then normalised to a ‘per one million good hits’ basis which is shown in Table 3, for four maternal plasma DNA samples, i.e. two normal (N1 and N2) and two Trisomy 21 pregnancies (T21/1 and T21/2):

TABLE 3

| | N1 | N2 | T21/1 | T21/2 |
|---|---|---|---|---|
| Total Good Hits | 1000000 | 1000000 | 1000000 | 1000000 |
| Ch21 | 12339 | 12313 | 13411 | 12852 |
| Ch1 | 80437 | 80494 | 80677 | 80536 |
| Ch2 | 90660 | 89925 | 90285 | 89869 |
| Ch3 | 71297 | 70812 | 70821 | 71644 |
| Ch4 | 65480 | 63059 | 63908 | 65153 |
| Ch5 | 64589 | 63208 | 63069 | 63318 |
| Ch6 | 61053 | 60399 | 60396 | 61597 |
| Ch7 | 52557 | 52240 | 52503 | 52725 |
| Ch8 | 51876 | 51510 | 50980 | 51633 |
| Ch9 | 39951 | 40152 | 39791 | 39881 |
| Ch10 | 49953 | 50806 | 50718 | 50179 |
| Ch11 | 46492 | 47391 | 47081 | 46829 |
| Ch12 | 45674 | 45220 | 45427 | 45610 |
| Ch13 | 35242 | 34199 | 34676 | 35010 |
| Ch14 | 31367 | 31357 | 31635 | 31298 |
| Ch15 | 32859 | 33217 | 33039 | 32502 |
| Ch16 | 26942 | 27881 | 27819 | 26861 |
| Ch17 | 26042 | 27153 | 27205 | 26782 |
| Ch18 | 28940 | 28691 | 28707 | 28588 |
| Ch19 | 13084 | 13925 | 14185 | 13537 |
| Ch20 | 22364 | 23077 | 22793 | 22254 |
| Ch22 | 11382 | 12389 | 12174 | 11791 |
| ChX | 38841 | 40081 | 38045 | 39025 |
| ChY | 573 | 497 | 653 | 525 |
| ChM | 7 | 2 | 3 | 3 |

Note that the four maternal samples shown in Tables 2 and 3 came from pregnancies where the fetus was confirmed as male, after the plasma samples from which these data were taken. This outcome could easily have been predicted from the data in Tables 2 and 3 in accordance with the second aspect of the invention.

To detect Trisomy 21 according to the invention, the ratio of Chromosome 21 hits relative to total hits on the other autosomes was calculated for each sample.

The values for N1, N2, T21/1 and T21/2 were as shown in Table 4:

TABLE 4
Ratios of Chromosome 21 hits relative to hits for other autosomes

| | N1 | N2 | T21/1 | T21/2 |
|---|---|---|---|---|
| Ratio | 0.0130 | 0.0130 | 0.0141 | 0.0136 |

The imbalances for the two Trisomy 21 cases are 1.0846 and 1.0462, respectively, and are therefore consistent with Trisomy 21 samples, where the fraction of fetal DNA is between 5% and 15%.

Using an extended data set to determine the value of the standard deviation, the z-scores for the 4 samples tested are respectively: −0.16 and −0.29, for the two normal cases and 5.50 and 2.55 for the two Trisomy 21 cases, indicating that the two Trisomy 21 cases were detected at approx 99% probability, or greater.

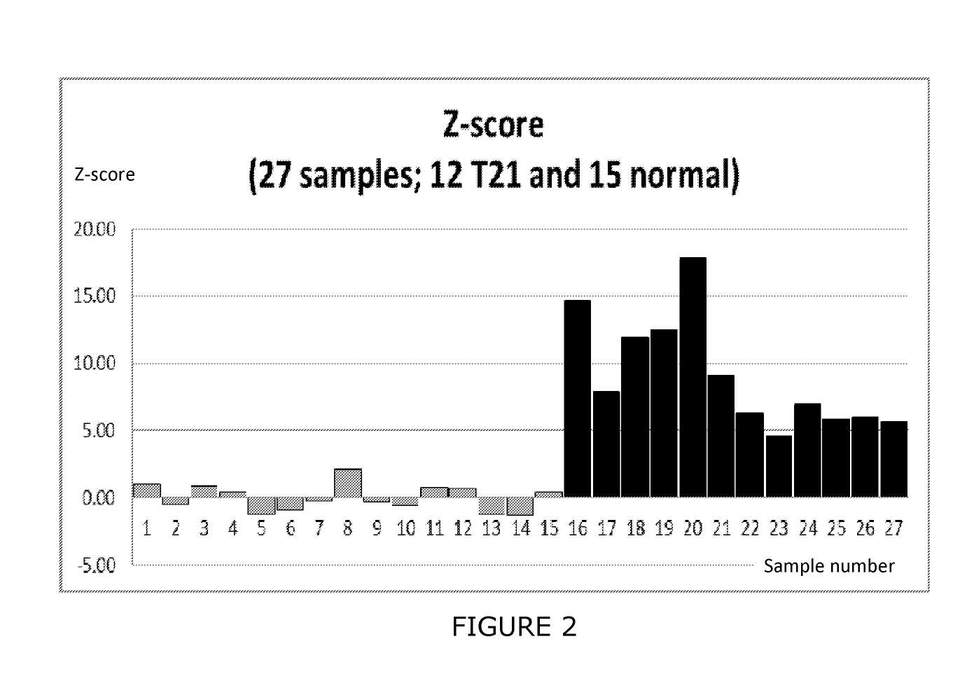

An analogous procedure was conducted on 27 blood plasma samples from normal pregnancies (samples 1-15) and blood plasma samples from Trisomy 21 pregnancies (samples 16-27) and the z-score results are shown in Table 5 and pictorially in FIG. 2 where it can be seen that the z-scores for the twelve Trisomy 21 cases range from 4.59 to 17.86 and between 2.09 and −1.31 for the fifteen normal cases. Table 5 also shows the differences in percentage of Chromosome 21 relative to other chromosomes, wherein a higher percentage can be seen for the twelve Trisomy 21 cases.

TABLE 5
Z-scores and % Ratios of Chromosome
21 in Normal and Trisomy 21 samples

| Sample Number | Z score | % Chromosome 21 |
|---|---|---|
| 1 (Normal) | 1.01 | 1.23 |
| 2 (Normal) | −0.52 | 1.22 |
| 3 (Normal) | 0.85 | 1.23 |
| 4 (Normal) | 0.48 | 1.23 |
| 5 (Normal) | −1.25 | 1.21 |
| 6 (Normal) | −0.91 | 1.21 |
| 7 (Normal) | −0.26 | 1.22 |
| 8 (Normal) | 2.09 | 1.24 |
| 9 (Normal) | −0.35 | 1.22 |
| 10 (Normal) | −0.56 | 1.22 |
| 11 (Normal) | 0.81 | 1.23 |
| 12 (Normal) | 0.70 | 1.23 |
| 13 (Normal) | −1.20 | 1.22 |
| 14 (Normal) | −1.31 | 1.21 |
| 15 (Normal) | 0.43 | 1.23 |
| 16 (Trisomy 21) | 14.67 | 1.34 |
| 17 (Trisomy 21) | 7.84 | 1.29 |
| 18 (Trisomy 21) | 11.98 | 1.32 |
| 19 (Trisomy 21) | 12.49 | 1.33 |
| 20 (Trisomy 21) | 17.86 | 1.37 |
| 21 (Trisomy 21) | 9.11 | 1.30 |
| 22 (Trisomy 21) | 6.29 | 1.27 |
| 23 (Trisomy 21) | 4.59 | 1.26 |
| 24 (Trisomy 21) | 6.99 | 1.28 |
| 25 (Trisomy 21) | 5.91 | 1.27 |
| 26 (Trisomy 21) | 5.97 | 1.27 |
| 27 (Trisomy 21) | 5.64 | 1.27 |

The data presented herein in Example 1, Table 5 and FIG. 2 demonstrate the clear ability of the method of the invention to be used to accurately and non-invasively diagnose Trisomy 21 in plasma DNA samples.

1. A method of detecting a fetal chromosomal abnormality in a biological sample obtained from a female subject, the method comprising the steps of:
(a) obtaining sequence data for nucleic acid molecules within the biological sample;
(b) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(c) measuring the ratio of the total number of matched nucleic acids assigned to a target chromosome relative to the total number of matched nucleic acids assigned to each of one or more reference chromosomes;
wherein a statistically significant difference in the measured ratio, relative to the ratio in a normal pregnancy, is indicative of a fetal abnormality in the target chromosome.

2. The method as defined in claim 1, wherein the target chromosome is chromosome 13, chromosome 18, chromosome 21, the X chromosome or the Y chromosome.

3. The method as defined in claim 1, wherein the fetal chromosomal abnormality is a fetal chromosomal aneuploidy, such as trisomy 13, trisomy 18 or trisomy 21.

4. (canceled)

5. (canceled)

6. A method of predicting the gender of a fetus within a pregnant female subject, the method comprising the steps of:
(a) obtaining a biological sample from the pregnant female subject;
(b) obtaining sequence data for nucleic acid molecules within the biological sample;
(c) performing a matching analysis between each nucleic acid sequence within the sequence data and a sequence which corresponds to a unique portion of a reference genome, such that each matched nucleic acid is assigned to a particular chromosome, or a part of said chromosome, within the reference genome, wherein said matching analysis generates an accuracy score for each base within each nucleic acid which corresponds to a base in the reference genome and a penalisation score for any insertions, deletions, ambiguities and/or substitutions, such that a match is assigned if the total score for each nucleic acid achieves a pre-determined score threshold; and
(d) measuring the ratio of the total number of matched nucleic acids assigned to the Y chromosome relative to the total number of matched nucleic acids assigned to each of one or more reference chromosomes;
wherein the presence of matched Y chromosome sequences above a pre-determined ratio is indicative of the presence of a male fetus and the presence of matched Y chromosome sequences below a pre-determined ratio is indicative of the presence of a female fetus.

7. The method as defined in claim 1, wherein the matching analysis is conducted by Bowtie2 or BWA-SW software or software employing Maximal Exact Matching techniques, such as BWA-MEM or CUSHAW2 software.

8. The method as defined in claim 1, wherein the matching analysis comprises the step of matching a nucleic acid to a pre-determined part of said chromosome within the reference genome.

9. The method as defined in claim 1, wherein the accuracy score is a positive score.

10. The method as defined in claim 9, wherein the positive score is +2 for each base within the nucleic acid which corresponds to a base in the reference genome.

11. The method as defined in claim 1, wherein the penalisation score for any insertions, deletions, ambiguities and/or substitutions is a reduced score, such as a negative score.

12. The method as defined in claim 11, wherein the negative score for a substitution is −6, the negative score for an ambiguity is −1 and the negative score for an insertion or deletion is −5 plus −3 for each residue within the insertion or deletion.

13. The method as defined in claim 1, wherein the minimum score threshold is defined by the following equation:
a+b*ln(L)
wherein a and b refer to scoring parameters determined to optimize matching accuracy and In refers to the natural logarithm of the read length (L).

14. The method as defined in claim 13, wherein a represents 20 and b represents 8.0.

15. The method as defined in claim 1, wherein the analysed nucleic acid sequence comprises from approximately 25 bp to approximately 250 bp.

16. The method as defined in claim 1, wherein the biological sample is maternal blood, plasma, serum, urine or saliva.

17. (canceled)

18. The method as defined in claim 1, which additionally comprises the step of collapsing duplicate reads from the sequence data obtained prior to the matching analysis step.

19. The method as defined in claim 1, which additionally comprises the step of normalizing or adjusting the number of matched hits based on the amount of fetal DNA within the sample.

20. The method as defined in claim 1, wherein the sequence data is obtained by a next generation sequencing platform or a sequencing platform which comprises use of a polymerase chain reaction; sequencing-by-synthesis; release of ions, such as hydrogen ions; semiconductor-based sequencing methodology; or nanopore-based sequencing methodology.

21.-25. (canceled)

26. The method as defined in claim 25, wherein said nanopore-based methodology comprises use of organic-type nanopores; or a nanopore constructed from a metal, polymer or plastic material.

27. (canceled)

28. The method as defined in claim 20, wherein the next generation sequencing platform is selected from: Roche 454 (i.e. Roche 454 GS FLX), Applied Biosystems' SOLiD system (i.e. SOLiDv4), Illumina's GAIIx, HiSeq 2000 and MiSeq sequencers, Life Technologies' Ion Torrent semiconductor sequencing platform, Pacific Biosciences' PacBio RS and Sanger's 3730xl.

29. (canceled)

30. The method as defined in claim 1, wherein the sequence data is obtained by Life Technologies' Ion Torrent Personal Genome Machine (Ion Torrent PGM) or multiplex capable iterations based upon the Life Technologies' Ion Torrent platform, such as an Ion Proton with a PI or PII Chip, and further derivative devices and components thereof.

31. (canceled)
